SEQUENCE LISTING (2 sequences)

SEQ ID NO: 1 - protein, 16 aa, Homo sapiens
KGRLLRLLRRRRRRRR

SEQ ID NO: 2 - protein, 11 aa, Homo sapiens
HHHRRRRRRRR

(19) United States
(12) Patent Application Publication
(10) Pub. No.: US 2018/0193472 A1
(43) Pub. Date: Jul. 12, 2018

(54) TRANSPORT PEPTIDES FOR TRANSCELLULAR DELIVERY OF BIOLOGICAL AND THERAPEUTIC AGENTS

(71) Applicant: THE JOHNS HOPKINS UNIVERSITY, Baltimore, MD (US)

(72) Inventors: Peter Charles Searson, Baltimore, MD (US); Honggang Cui, Lutherville-Timonium, MD (US); Kalina Hristova, Baltimore, MD (US); Ran Lin, Baltimore, MD (US); Alexander Komin, Glen Burnie, MD (US)

(21) Appl. No.: 15/860,710

(22) Filed: Jan. 3, 2018

Related U.S. Application Data
(60) Provisional application No. 62/443,258, filed on Jan. 6, 2017.

(51) Int. Cl. A61K 47/62; A61K 49/00; A61P 35/00; A61P 25/18; A61P 25/08; A61P 25/24; A61P 25/00; A61P 25/20; A61P 25/28; A61P 25/16

(52) U.S. Cl. CPC A61K 47/62; A61K 49/0056; A61P 35/00; A61P 25/18; A61P 25/16; A61P 25/24; A61P 25/00; A61P 25/20; A61P 25/28; A61P 25/08

(57) ABSTRACT

Transport peptides with extraordinary ability to effectively transcellular transport cargo(s) across biological barriers have been identified. While the cargo itself shows a very limited ability to cross the biological barrier, the conjugated form with the transport peptide of the present invention enhances the rate of cargo transport across a biological barrier by approximately 30 times.

### CROSS-REFERENCE TO PRIOR APPLICATIONS

This application claims the benefit of U.S. Provisional Application No. 62/443,258, filed Jan. 6, 2017, which is hereby incorporated by reference for all purposes as if fully set forth herein.

### INCORPORATION-BY-REFERENCE OF MATERIAL SUBMITTED ELECTRONICALLY

This application contains a sequence listing. It has been submitted electronically via EFS-Web as an ASCII text file entitled “P13347-02_ST25.txt.” The sequence listing is 623 bytes in size, and was created on Jan. 4, 2016. It is hereby incorporated by reference in its entirety.

### BACKGROUND OF THE INVENTION

Cell-penetrating peptides (CPPs) are short peptides having 5 to 30 amino acids and are capable of entering mammalian cells. CPPs can be categorized as cationic, hydrophobic, and amphipathic, based on their sequence. Cationic peptides are positively charged due to the presence of arginines and lysines. A large number of cationic variations of CPPs have been synthesized with the famous examples being truncated versions of HIV-TAT (amino acids 47-57), oligoarginines, and penetratin. The proposed modes of entry of CPPs into the cells are direct penetration of cell lipid membrane and endocytotic receptor-independent pathways, although the mechanisms are not fully understood. While CPPs have been demonstrated to deliver cargo (e.g. quantum dots, therapeutics, genetic material, liposomes, etc.) intracellularly, a very limited information exists on the ability of CPPs to transport cargo transcellularly across a biological barrier. Transcellular transport involves the transport of cargo across the cellular layers, such that cargo is internalized on one side of cells and exited through another. In contrast, the paracellular transport involves cargo diffusion across the junctions between the interconnected cells and does not involve entry inside the cells. Paracellular transport is very limited, or virtually non-existent, when the integrity of junctions between the cells is high. This is especially the case in the blood-brain barrier, which separates circulating blood from the brain preventing many compounds within a subject from entering a subject's brain. Consequently, a large number of important CNS disorders are ineffectively treated because drugs are unable to reach the brain of patients inflicted with such disorders. Specifically, less than 2% of the small molecules and almost none of the large molecules, including pharmaceutical agents, cross the blood-brain barrier. Drug delivery mechanisms useful for the delivery of small molecule therapeutics, biopharmaceuticals, siRNA genes, and imagining agents across the blood-brain barrier for the purposes of scientific research, diagnosis, and treatment of disorders must be identified to help patients with neurological disease.

### SUMMARY OF THE INVENTION

The present invention provides compositions and methods to facilitate delivery of small-molecule therapeutics, biopharmaceuticals, siRNA, genes, and imaging agents across biological membranes such as, the blood-brain barrier, epithelial cells, and endothelial cells (in vivo and in vitro) for scientific research, diagnosis, and treatment of disorders in subjects.

In accordance with an embodiment, the present invention provides a composition of formula (I);

C-T (I)

wherein C is one or more cargo each having a molecular weight in the range of 50 Da to 50,000 Da; and wherein T is a transport peptide having amino acid SEQ ID NO: 1; or a pharmaceutically acceptable salt, solvate, or stereoisomer thereof. Suitable cargo is selected from the group comprising: a small chemical therapeutic, a biopharmaceutical, siRNA gene, an imaging agent, or combination thereof. Preferably, the cargo has a molecular weight in the range of 100 Da to 25,000 Da or in the range of 100 Da to 10,000 Da.

In accordance with an embodiment, the present invention provides a method of treatment of disease in a subject comprising administering an effective amount of a composition of formula I:

C-T (I)

wherein C is one or more cargo each having a molecular weight in the range of 50 Da to 50,000 Da; and wherein T is a transport peptide having amino acid SEQ ID NO: 1; or a pharmaceutically acceptable salt, solvate, or stereoisomer thereof. The composition of formula I can be used to treat disease including, neurological disease; cancer; a disease that requires the transport of the cargo across a blood-brain barrier within a subject; a CNS disorder selected from the group comprising Schizophrenia, Epilepsy, Depression, Chronic Pain, Insomnia, ADHD, Alzheimer's disease, Huntington's disease, Parkinson's disease, A.L.S., Stoke, Brain Cancer, Multiple Sclerosis, Brain infections, and traumatic brain injury; disease that requires the transport of the cargo across an epithelial cell and a disease that requires the transport of the cargo across an endothelial cell, for example. The treatment of disease is based on the cargo attached to the transport peptide. Compositions of the present invention are able to deliver one or more composition(s) to a target such as the brain. One advantage of using a transport peptide of the present invention is that it can potentially be used for the delivery of therapeutics across a healthy undisrupted blood brain barrier, or any biological barrier, without damaging it.

In accordance with an embodiment, the present invention provides a method of transporting cargo across a biological barrier comprising the following steps: providing a biological barrier having a top and bottom; providing the compositions of the present invention; contacting the top of the biological barrier with the compositions of the present invention; transporting the composition of the present invention through the barrier and out of the bottom of the biological barrier. The composition of the present invention is moved through the cells making up the barrier by transcellular transport. The biological barrier maybe endothelial cells, such as the endothelial cells of the blood-brain barrier, and/or epithelial cells. The method of transporting cargo across a biological barrier may occur in vitro or in vivo.

In accordance with an embodiment, the present invention provides pharmaceutical composition comprising a compound, salt, solvate, or stereoisomer of any one of the compounds of formula I, as set forth above, and at least one or more other drug delivery system, such as liposomes, and/or scaffolds that when attach to the pharmaceutical composition, and injected into a body of a subject, enables the release of the pharmaceutical composition in a controlled manner.

In another embodiment, the present invention provides a method of transporting cargo across a biological membrane, comprising administering to the subject an effective amount of a composition, salt, solvate, or stereoisomer of any one of the compositions of formula I, as set forth above.

In another embodiment, the present invention provides a method of transporting cargo across an undisrupted blood-brain-barrier, comprising administering to the subject an effective amount of a composition, salt, solvate, or stereoisomer of any one of the compositions of formula I, as set forth above

In accordance with an embodiment, the present invention provides a method of treatment of one or more disease(s) in a subject comprising administering an effective amount of a composition of formula I.

### BRIEF DESCRIPTION OF THE DRAWINGS

FIG. 1 illustrates one transport peptide of the present invention, the CL peptide.

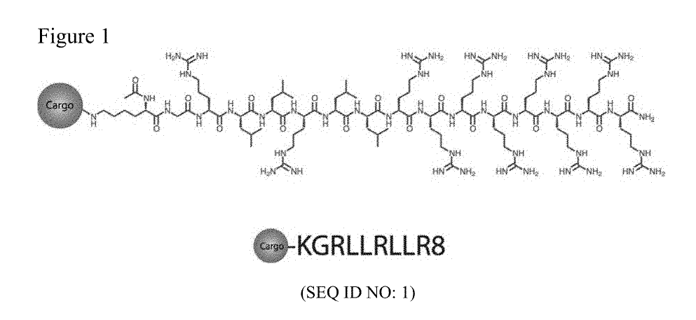

FIG. 2 illustrates the apparent permeabilities (Papp) of: 1) A transport peptide of the present invention, CL, attached to 5-Carboxyfluorescein designated as [5-FAM] CL; 2) 5-Carboxyfluorescein attached to H3R8 designated as [5-FAM] H3R8; 3) 5-Carboxyfluorescein designated as 5-FAM, and 4) Lucifer Yellow designated as LY. The permeability of all four of these compounds across a monolayer of MDCKII cells, grown on a porous membrane, is described in the Examples.

FIG. 3 illustrates the percent change in MDCKII transepithelial electrical resistance (TEER) of the MDCKII cells after the permeability experiment described in FIG. 2 and the Examples. As in FIG. 1, the transport peptide of the present invention, CL, attached to 5-Carboxyfluorescein is designated as [5-FAM] CL; 2) 5-Carboxyfluorescein attached to H3R8 is designated as [5-FAM] H3R8; 3) 5-Carboxyfluorescein is designated as 5-FAM, and 4) Lucifer Yellow is designated as LY.

FIG. 4A-B illustrates a) the amino acid sequence of a transport peptide of the present invention known as the CL peptide and b) the amino acid sequence of a H3R8 peptide.

### DETAILED DESCRIPTION OF THE INVENTION

In accordance with the present invention, the inventors have determined that the compounds of Formula I, may be clinically useful for treating or preventing disease, such as CNS disorder including Schizophrenia, Epilepsy, Depression, Chronic Pain, Insonmia, ADHD, Alzheimer's disease, Huntington's disease, Parkinson's disease, A.L.S., Stoke, Brain Cancer, Multiple Sclerosis, Brain infections, and traumatic brain injury.

In an embodiment, the present invention provides a composition of Formula I:

C-T (I)

wherein C is one or more cargo element having a molecular weight from 100 Da to 900 Da; and wherein T is a peptide selected from the group of amino acid SEQ ID NOS: 1-3; or a pharmaceutically acceptable salt, solvate, or stereoisomer thereof.

The transport peptides of the present invention were identified by modifying peptide sequences and testing these peptides in a Transwell assay measuring in vitro transcellular transport. The Transwell assay consists of two chambers separated from each other by a permeable porous membrane. Cells, most commonly canine kidney epithelial MDCK cells or human intestinal epithelial Caco-2 cells, are grown on the membrane and represent a model biological barrier. The drug, or peptide of interest, is added to the input chamber and the concentration in the receiver chamber is measured after a period of time. The rate at which the drug or peptide permeates across the cells (and the porous membrane) is the rate of drug's concentration increase in the receiver chamber after a period of time. The rate can be calculated and expressed in terms of apparent permeability (Papp), which takes into account the input concentration, the rate of concentration increase in the receiver well, solution volume in the receiver well, and the area of the cell monolayer. Substances with low permeability generally have Papp values on the order of 10⁻⁷ cm/s, and substances with high permeabilities generally have Papp values on the order of 10⁻⁵ cm/s or higher. There are several published reports where the CPPs were investigated for their ability to deliver cargo transcellularly using a Transwell assay: Violini et al reported low permeabilities of 3.1×10⁻⁸ cm/s and 7.4×10⁻⁸ cm/s for TC-radiolabeled Tat 48-57 across Caco-2 and MDCK II cells, respectively.

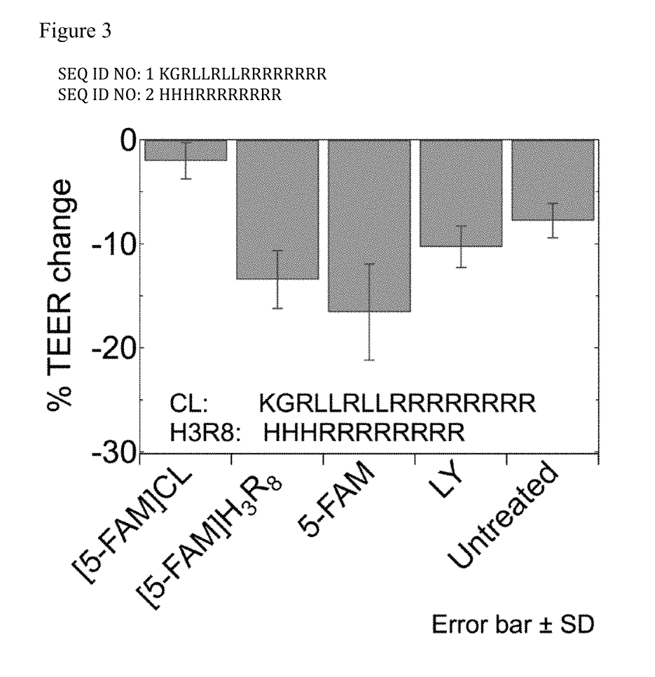

The inventors identified cationic transport peptides that are capable of transcellular delivery of cargo across a monolayer of MDCKII cells grown on a porous membranes. One embodiment of the invention is a CL peptide, conjugated to a fluorescent dye 5-FAM (fluorescein), has a Papp value of (1.3±0.2)×10⁻⁵ cm/s, which approaches the permeability rates of the fast-permeating molecules into the brain (Papp ≈10⁻⁵ to 10⁻⁴ cm/s). The CL peptide, conjugated to 5-FAM, enhanced 30-fold the rate of permeation of 5-FAM across the biological barrier. This is especially significant because MDCKII cells are virtually impermeable to 5-FAM (Papp=(4.1±0.6)×10⁻⁷ cm/s) unless it is conjugated to the novel peptide. In addition, the CL peptide was able to deliver 5-FAM without compromising the junctional integrity of the model cell monolayer. Junctional integrity of the monolayer can be measured by transepithelial electrical resistance (TEER), which signifies resistance to the flow of ions across a cell monolayer. If the tight junctions between the cells are compromised, the TEER decreases. The CL peptide did not significantly affect the TEER when compared to untreated cells and negative controls as shown in FIG. 3. This suggests that the transport peptides of the present invention delivered the cargo transcellularly, rather than paracellularly.

### Applications of Cell Penetrating Peptide of the Present Invention

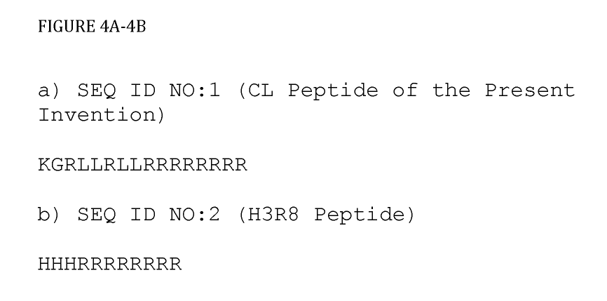

The compositions of Formula (I) of the present invention, comprising cargo and a transport peptide. Amino acid sequences of one transport peptide, CL, of the present invention is described in SEQ ID NO: 1 described in FIG. 4a). Cargo is defined as one or more compounds having a molecular weight in the range beginning with a first molecular weight to a second molecular weight such as 50 Da-50,000 Da, 100 Da-45,000 Da, 100 Da-40,000 Da, 100 Da-35,000 Da, 100 Da-30,000 Da, 100 Da-25,000 Da, 100 Da-20, 000 Da, 100 Da-15, 000 Da, 100 Da to 10,000 Da, 100 Da-5,000 Da, 100 Da,-1,000 Da, 100 Da-900 Da, 150 Da -850 Da, 200 Da-800 Da, 250 Da-750 Da, 300 Da-700 Da, or 350 Da-650 Da, or any ranges in between resulting from any combination of a first molecular weight with a second molecular weight such as 150 Da -700 Da or 200 Da-650 Da. Preferred cargo include compounds such as chemical therapeutics, protein-based drugs (biologics), nucleic acids (siRNA, mRNA, shRNA as examples) and imaging agents or any combinations thereof. The cargo transport peptide complex of formula (I) enable the transcellular delivery of cargos across epithelial barriers, endothelial biological barriers, like the blood-brain barrier endothelial cells, and to the desired targets, such as the brain. The transport peptides of the present invention will help deliver pharmaceutical agents in patients having CNS disorder including Schizophrenia, Epilepsy, Depression, Chronic Pain, Insonmia, ADHD, Alzheimer's disease, Huntington's disease, Parkinson's disease, A.L.S., Stoke, Brain Cancer, Multiple Sclerosis, Brain infections, and traumatic brain injury. The transport peptide can potentially improve the delivery of existing drugs such as the ones that are known to be exported by active efflux pumps specifically MDR-1/PgP active efflux substrates including doxorubicin, paclitaxel. A lot of candidate drugs show promise in treating CNS disorders during the drug discovery stage, but cannot reach the brain and achieve useful therapeutic concentrations. Combining these candidate drugs to the transport peptide of the present invention may enhance their therapeutic effect and the development of commercial drugs. Compositions of the present invention are able to deliver one or more composition(s) to a target such as the brain. One advantage of using a transport peptide of the present invention is that it can potentially be used for the delivery of cargo (therapeutics) across a healthy undisrupted blood-brain barrier, or any biological barrier, without damaging it. Many cargo/compounds may be attached to one or more transport peptide (s) of the present invention to create new therapeutics to treat or prevent disease. One method of treatment of disease in a subject begins by administering an effective amount of a composition of Formula I:

C-T (I)

wherein C is one or more cargo element (s) having a molecular weight from 100 Da to 900 Da; and wherein T is a peptide selected from the group of amino acid SEQ ID NO: 1; or a pharmaceutically acceptable salt, solvate, or stereoisomer thereof. In addition the compositions of Formula (I) may be combined with other drug delivery systems such as liposomes.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Permeability Studies Across Biological Membranes

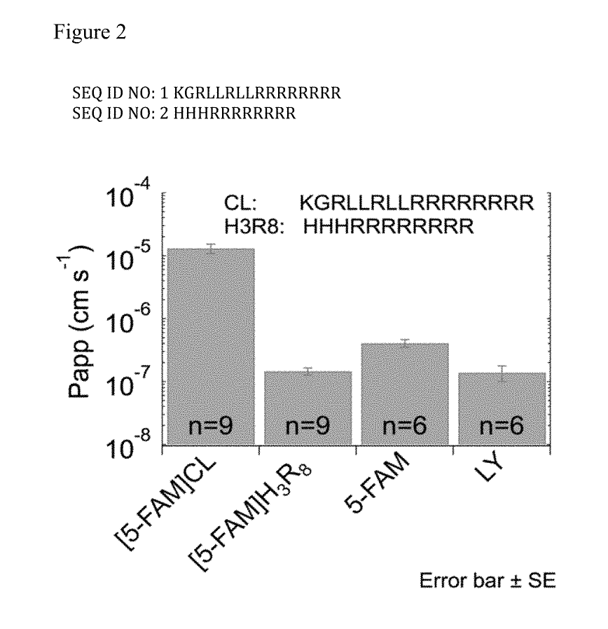

The apparent permeabilities (Papp) of four compounds were determined across a monolayer of MDCKII cells, grown on a porous membrane. The four compounds were the following and are represented in FIGS. 2-3: 1) a transport peptide of the present invention, CL, attached to 5-Carboxyfluorescein (MW: 376.3) and designated as [5-FAM]CL peptide, 2) 5-Carboxyfluorescein attached to H3R8 ([5-FAM] H3R8 MW: 2,263.5 and the H3R8 peptide is shown in FIG. 4b) 3) 5-Carboxyfluorescein designated as 5-FAM and 4) Lucifer Yellow designated as LY (MW:457.25, Product Number L-453, Life Technologies). The following concentrations of [5-FAM] CL, [5-FAM] H3R8, 5-FAM, and LY compound were applied to the monolayer of MDCKII cells at approximately 0.2 μM, 0.4-0.9 μM, 4 μM, and 100 μM, respectively. The peptides and dyes were resuspended in HEPES- and glucose-supplemented 1×HBSS buffer. The permeability experiments were conducted for 1 hour at 37° C. Error bars represent standard error (SE). CL and H3R8 are conjugated to 5-FAM at the N-termini. H3R8 is based on oligoarginine CPPs that has been reported to deliver cargo intracellularly. LY is used to assess monolayer integrity (Papp≤10̂−6 cm/s verifies monolayer integrity). The permeability of LY is on the order of 10̂−7 cm/s and is thus consistent with literature values for a monolayer with good barrier properties. [5-FAM] CL has Papp that is 30 times higher than 5-FAM, indicating that it was able to significantly enhance the delivery of 5-FAM across a monolayer of MDCKII cells. On the other hand, a cationic cell-penetrating peptide H3R8 was not capable to deliver 5-FAM across a monolayer of MDCKII cells.

### Transepithelial Electrical Resistance (TEER) Measurements

To determine whether or not the four compounds used in the permeability study resulted in damage to the integrity of the monolayer of MDCKII cells, transepithelial electrical resistance measurements were taken on the MDCKII cells before and after the permeability study to determine the % TEER change. FIG. 3 illustrates the % TEER change in the monolayer of MDCKII cells. Error bars represent SD with n=3 for all except LY, n=6. As illustrated in FIG. 3, treating the MDCKII cells with [5-FAM] CL and [5-FAM] H3R8 did not result in a significant % TEER decrease after the permeability experiment compared to LY treatment and untreated cells. The data indicates the high permeability rates, observed for a transport peptide of the present invention, [5-FAM] CL, should be attributed to a transcellular mode of transport.

1. A composition comprising a compound of formula (I), or salt, solvate, or stereoisomer of any one of the compounds of formula (I);
C-T (I)
wherein C is one or more cargo having a molecular weight in the range of 50 Da to 50,000 Da; and T comprises a peptide having amino acid SEQ ID NO: 1 or a functional part thereof.

2. The composition of claim 1 wherein the cargo is selected from the group comprising: a small chemical therapeutic, a biopharmautical, siRNA gene, an imaging agent, or combination thereof.

3. The composition of claim 1 wherein the cargo has a molecular weight in the range of 100 Da to 25,000 Da.

4. The composition of claim 1 wherein the cargo has a molecular weight in the range of 100 Da to 10,000 Da.

5. The composition of claim 1 wherein T is SEQ ID NO: 1.

6. A method of treatment or prevention of a disease in a subject comprising administering to a subject an effective amount of a composition comprising a compound of formula (I), or a salt, solvate, or stereoisomer of any one of the compounds of formula (I):
C-T (I)
wherein C is one or more cargo having a molecular weight in the range of 50 Da to 50,000 Da; and T comprises a peptide having amino acid SEQ ID NO: 1 or a functional part thereof; and
treating or preventing a disease in a subject.

7. The method of claim 6 wherein the cargo has a molecular weight in the range of 100 Da to 25,000 Da.

8. The method of claim 6 wherein the cargo has a molecular weight in the range of 100 Da to 10,000 Da.

9. The method of claim 6, wherein the disease is a neurological disease.

10. The method of claim 6, wherein the disease is cancer.

11. The method of claim 6, wherein the treatment of the disease requires the transport of the cargo across a blood-brain barrier within a subject.

12. The method of claim 6 having an additional step of delivering the composition to a target.

13. The method of claim 12, wherein the target is the brain.

14. The method of claim 6, wherein the disease is a CNS disorder selected from the group comprising Schizophrenia, Epilepsy, Depression, Chronic Pain, Insomnia, ADHD, Alzheimer's disease, Huntington's disease, Parkinson's disease, A.L.S., Stroke, Brain Cancer, Multiple Sclerosis, Brain infections, and traumatic brain injury.

15. The method of claim 6, wherein the treatment of the disease requires the transport of the cargo across an epithelial cell.

16. The method of claim 6 wherein the treatment of the disease requires the transport of the cargo across an endothelial cell.

17. A method of transporting cargo across a biological barrier comprising the following steps:
a. providing a biological barrier;
b. providing a composition comprising a compound of formula (I), or a salt, solvate, or stereoisomer of any one of the compounds of formula (I):
C-T (I)
wherein C is one or more cargo having a molecular weight in the range of 50 Da to 50,000 Da; and T comprises a peptide having amino acid SEQ ID NO: 1 or a functional part thereof;
c. contacting the biological barrier with the composition;
d. transporting the composition across the biological barrier.

18. The method of claim 17 wherein the method of transporting cargo across a biological membrane is in vivo.

19. The method of claim 17 wherein the biological barrier are endothelial cells.

20. The method of claim 19 wherein the biological barrier is the blood-brain barrier.

21. The method of claim 17 wherein the biological barrier are epithelial cells.

22. The method of claim 17 having an additional step of delivering the composition to a target.

23. The method of claim 22, wherein the target is the brain.

24. The method of claim 17 wherein the transport of the composition is through a transcellular transport.

25. A pharmaceutical composition comprising a compound of formula (I), or a salt, solvate, or stereoisomer of any one of the compounds of formula (I),
C-T (I)
wherein C is one or more cargo having a molecular weight in the range of 50 Da to 50,000 Da; and T comprises a peptide having amino acid SEQ ID NO: 1 or a functional part thereof; and
at least one or more other drug delivery system.

26. The pharmaceutical composition of claim 25 wherein the one or more drug delivery system is selected from the group comprising liposomes, scaffolds, or a combination thereof.

27. The pharmaceutical composition of claim 26, wherein the drug delivery system releases the pharmaceutical composition in a controlled manner.
